(19) United States
(12) Patent Application Publication
(10) Pub. No.: US 2011/0106181 A1
(43) Pub. Date: May 5, 2011

(54) ADJUSTABLE SADDLE FOR A BONE ANCHOR

(75) Inventors: William A. Rezach, Atoka, TN (US); Suzanne M. Nielsen, San Clemente, CA (US)

(73) Assignee: Warsaw Orthopedic, Inc., Warsaw, IN (US)

(21) Appl. No.: 12/609,984

(22) Filed: Oct. 30, 2009

(51) Int. Cl. A61B 17/86

(52) U.S. Cl. 606/308

(57) ABSTRACT

A bone anchor is provided, which may be used in cervical, thoracic, lumbar or sacral areas of the spine or other orthopedic locations. The anchor includes an anchoring portion, a receiving portion, and an internal saddle that is rotatable with respect to the receiving portion at least to a degree. The anchoring portion is attachable to a bone. A rod or other elongated support member is received in the receiving portion in contact with the saddle. The rod and saddle may be rotated for variability substantially in a plane, and an engaging member is engaged to the receiving portion to lock the rod within the receiving portion.

### BACKGROUND

The present invention concerns bone anchors and fixation systems, particularly useful for engagement to vertebrae. In a particular embodiment, the invention contemplates a bone anchor with an adjustable saddle that is engageable to an elongate connecting element, such as a spinal rod, positionable along the spinal column.

Several techniques and systems have been developed for correcting and stabilizing the spine and for facilitating fusion at various levels of the spine. In one type of system, an elongated rod is disposed longitudinally along the length of the spine or several vertebrae of the spinal column. The rod may be bent to correspond to the normal or desired curvature of the spine in the particular region being instrumented. For example, the rod can be bent or angled to form a normal kyphotic curvature for the thoracic region of the spine, or a lordotic curvature for the lumbar region. In accordance with such a system, the rod is engaged to various vertebrae along the length of the spinal column by way of a number of fixation elements. A variety of fixation elements can be provided which are configured to engage specific portions of the vertebra. For instance, one such fixation element is a hook that is configured to engage the lamina of the vertebra. Another type of fixation element is a spinal screw which can be threaded into various aspects of the vertebral bone, such as the pedicle.

In one typical procedure utilizing a bendable or angled rod, the rod is situated on one or opposite sides of the spine or spinous processes. A plurality of bone screws are threadingly engaged to several vertebral bodies, such as to the pedicles of these vertebrae. The rod(s) are connected or affixed to the plurality of bone screws to apply and maintain corrective and stabilizing forces to the spine.

The bone anchors in spinal procedures can have channels for the elongated rod or other member that, in some bone anchors, open upward, i.e. directly away from the bone to which the anchor is attached. Other bone anchors utilize channels that open along the medial or lateral side of the anchor to receive the rod. The contouring and/or angling of the rod through the channels of these bone anchors can result in a less than optimal fit between the anchor and the rod, creating stress concentrations in the rod and/or anchor. Furthermore, many bone anchor designs utilize several small components to assemble the various parts of the bone anchor together, making manufacture and assembly of the bone anchors expensive and time consuming. Additional improvements in the bone anchor and rod interface in spinal systems are still needed.

### SUMMARY

A bone anchor is provided, which may be used in cervical, thoracic, lumbar or sacral areas of the spine or other orthopedic locations. The anchor includes an anchoring portion, a receiving portion, and an internal saddle within a channel defined by the receiving portion. A rod or other elongated support member is received in the receiving portion in contact with the saddle.

According to one aspect, a bone anchor apparatus for attachment of an elongated spinal rod to a vertebra is provided. The bone anchor apparatus includes a bone engaging portion having a threaded shaft extending along a longitudinal axis from a distal end to a neck at a proximal end of the threaded shaft. A receiving portion includes a lower leg connected to the neck of the bone anchor portion, and the lower leg has a foot portion extending proximally from one end of the lower leg. The receiving portion also includes an upper leg opposite the lower leg that extends transversely to the longitudinal axis. The receiving portion also includes an intermediate portion opposite the foot portion that extends between the upper leg and the lower leg. The intermediate portion includes a blind hole extending therein. The receiving portion also includes a channel extending through the receiving portion substantially perpendicular to the longitudinal axis and into which the spinal rod can be placed. The receiving portion defines a mouth opposite the intermediate portion for introduction of the spinal rod into the channel. The blind hole opens into the channel and the foot portion defines a lip that extends from the lower leg toward the mouth. The bone anchor also includes a saddle connected to the receiving portion so that at least a portion of the saddle is within the channel of the receiving portion. The saddle includes a proximal end with a post portion extending into the blind hole of the intermediate portion of the receiving portion. The saddle further includes a seat portion extending between the proximal end and a lateral end. The lateral end is positioned in the channel adjacent to the lip of the foot portion with the post portion in the blind hole of the intermediate portion to secure the saddle in the channel. The seat portion defines a concavely curved inner surface along the channel.

According to another aspect, a bone anchor apparatus for attachment of an elongated spinal rod to a vertebra includes a bone engaging portion extending along a longitudinal axis between a distal end and a proximal end and a receiving portion having a lower leg extending from the proximal end of the bone engaging portion. The receiving portion includes at least one intermediate leg extending from a first side of the lower leg along the longitudinal axis and a foot portion extending proximally along the longitudinal axis from a second side of the lower leg opposite the intermediate portion. The receiving portion includes a channel defined at least in part by the lower leg, the intermediate portion and the foot portion. The channel extends substantially perpendicular to the intermediate portion and the longitudinal axis, and the receiving portion further includes a cavity extending from the foot portion and across the lower leg to a blind hole extending into the intermediate portion. The receiving portion includes a saddle that has a post portion and a seat portion with the post portion projecting laterally from the seat portion adjacent to a proximal end of the saddle. The seat portion extends from the post portion along a substantially cylindrically shaped concave inner surface to a second end of the saddle. The seat portion is positioned at least in part in the cavity with the second end in abutting engagement with the foot portion with the post portion in the blind hole.

According to another aspect, a bone anchor apparatus for attachment of an elongated spinal rod to a vertebra includes a bone engaging portion extending along a longitudinal axis between a distal end and a proximal end and a receiving portion having a lower leg extending from the proximal end of the bone engaging portion. The receiving portion includes at least one intermediate leg extending from a first side of the lower leg along the longitudinal axis and a foot portion extending proximally along the longitudinal axis from a second side of the lower leg opposite the intermediate portion. The receiving portion also includes a channel defined at least in part by the lower leg, the intermediate portion and the foot portion. The channel extends substantially perpendicular to the intermediate portion and the longitudinal axis. The anchor further includes a saddle including a post portion and a seat portion. The post portion projects laterally from the seat portion adjacent to a proximal end of the saddle. The seat portion extends from the post portion along a substantially cylindrically shaped concave inner surface to an abutting surface at a second end of the saddle. The saddle is bendable to a reduced size configuration to position the saddle in the channel with the post portion in the blind hole and the abutting surface adjacent to the foot portion. The saddle is resilient to contact the abutting surface with a proximally extending lip of the foot portion after the saddle is positioned in the channel to maintain the post portion in the blind hole.

These and other aspects are discussed further below.

### BRIEF DESCRIPTION OF THE FIGURES

FIG. 1 is a side view of an embodiment of a side-loading bone anchor with a saddle mounted therein.

FIG. 2 is a side view of the bone anchor shown in FIG. 1, rotated ninety degrees with respect to FIG. 1.

FIG. 3 is a side view of the bone anchor shown in FIG. 1, rotated 180 degrees with respect to FIG. 1.

FIG. 4 is a longitudinal cross-sectional view of the bone anchor shown in FIG. 1, taken along line 4-4 in FIG. 3.

FIG. 5 is a side view of a portion of the bone anchor shown in FIG. 1.

FIG. 6 is an exploded view of the bone anchor in the orientation of FIG. 2.

FIG. 7 is a longitudinal cross-sectional view of the exploded bone anchor of FIG. 6.

FIG. 8 is a top view of an embodiment of a saddle removed from the apparatus shown in FIG. 1.

FIG. 9 is a side view of the saddle shown in FIG. 8.

FIG. 10 is a rear view of the saddle shown in FIG. 8.

FIG. 11 is a front view of the saddle shown in FIG. 8.

FIG. 12 is a side view of another embodiment of a side-loading bone anchor with a saddle mounted therein.

FIG. 13 is a side view of the bone anchor shown in FIG. 12, rotated ninety degrees with respect to FIG. 12.

FIG. 14 is a side view of the bone anchor shown in FIG. 12, rotated 180 degrees with respect to FIG. 12.

FIG. 15 is the view of the bone anchor of FIG. 14 with the saddle removed.

FIG. 16 is a longitudinal cross-sectional view of the bone anchor shown in FIG. 12, taken along line 16-16 in FIG. 12.

FIG. 17 is a side view of a proximal portion of the bone anchor shown in FIG. 13.

FIG. 18 is a cross-sectional view of the bone anchor portion of FIG. 17 taken along line 18-18 of FIG. 17.

FIG. 19 is an exploded elevation view of the bone anchor of FIG. 13.

FIG. 20 is a longitudinal cross-sectional view of the exploded bone anchor of FIG. 19.

FIG. 21 is a perspective view of a receiver of the bone anchor of FIG. 12.

FIG. 22 is a perspective view of the saddle of the bone anchor of FIG. 12.

FIG. 23 is a perspective view looking toward the rear of the saddle shown in FIG. 22.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein, being contemplated as would normally occur to one skilled in the art to which the invention relates.

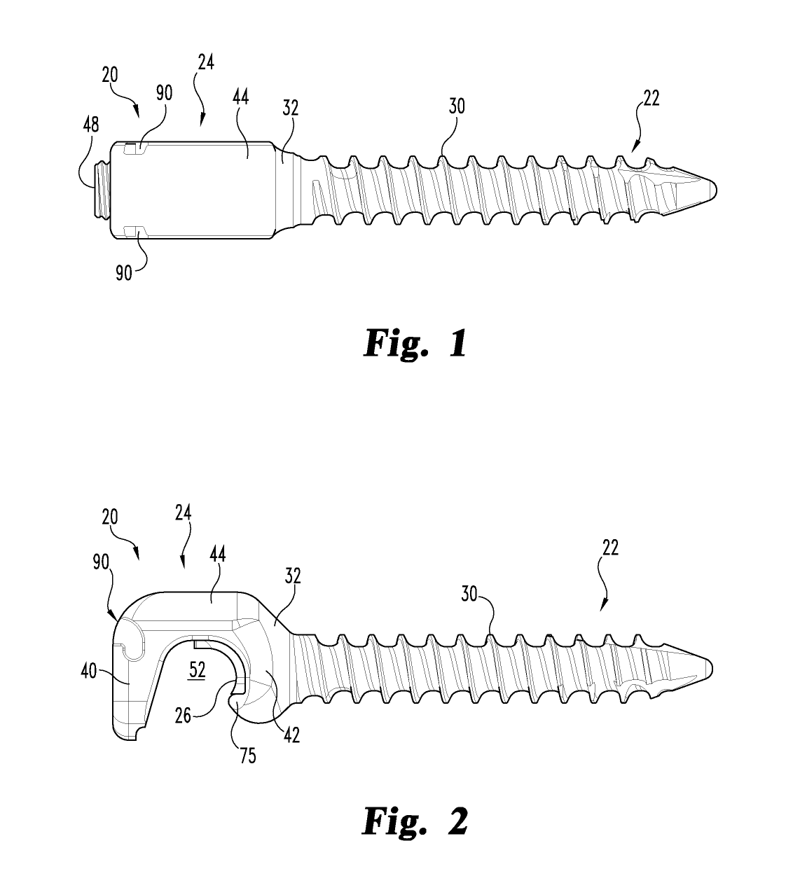

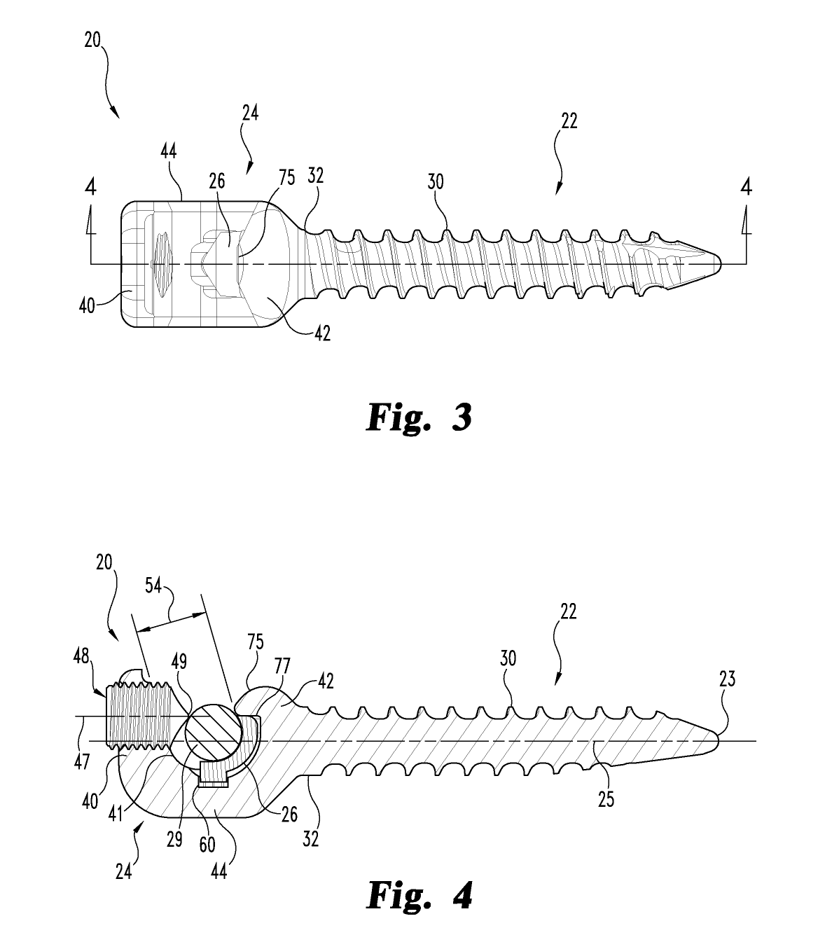

Referring to FIGS. 1-4, there is shown an embodiment of a side-loading bone anchor 20, it being understood that the aspects of the invention also have application with top-loading, bottom loading, and oblique loading type bone anchors. Bone anchor 20 includes a distal bone engaging portion 22 configured for attachment to a vertebra, such as cervical, thoracic, lumbar and or sacral bone structures, or other bones or tissues in the body of a patient. Aspects of bone anchor 20 described herein can be included with bone engaging portion 22 configured as a bone screw, vertebral hook, bone clamp, and or other suitable bone engaging arrangement.

Bone anchor 20, in the embodiment shown in FIGS. 1-4, includes an elongated bone engaging portion 22 extending from a distal end 23 along a longitudinal axis 25, a receiving portion 24 at a proximal end of bone engaging portion 22, and a saddle 26 in receiving portion 24. Bone engaging portion 22 is shown as an elongated shaft having one or more threads 30 along at least a portion thereof. Thread 30 may be a cancellous thread with the shaft sized and configured for implantation into a vertebra or other bone. Bone engaging portion 22 and/or thread 30 may be self-tapping, self-drilling, continuous, intermittent, of multiple thread forms, or other appropriate configurations. A neck 32 at a proximal end of bone engaging portion 22 extends between bone engaging portion 22 and receiving portion 24. Neck 32 may be entirely proximal of thread 30, or may also include a portion of thread 30. In the illustrated embodiment, bone engaging portion 22 is integrally joined to receiving portion 24, as by unitary formation or construction or by solid attachment, with neck 32. Other embodiments contemplate a variable angle or pivoting type connection between receiving portion 24 and bone engaging portion 22.

Referring now additionally to FIGS. 5-7, the depicted embodiment of receiving portion 24 is substantially C-shaped, having an upper leg 40, a lower leg 42 including foot portion 75 extending from one end thereof, and an intermediate portion 44 joining upper and lower legs 40, 42 opposite of foot portion 75. Other embodiments contemplate, for example, a U-shaped opening with a pair of intermediate portions extending proximally from lower leg 42. Receiving portion 24 defines a mouth 54, as referenced in FIG. 4, between upper leg 40 and foot portion 75 that is opposite intermediate portion 44. Mouth 54 opens into channel 52 extending through receiving portion 24, with channel 52 extending in an orthogonal relationship to longitudinal axis 25. Upper leg 40 has a threaded aperture 46 into which an engaging member 48 can be threadingly engaged. Aperture 46 extends along a central longitudinal axis 47 defining the direction of advancement of engaging member 48 into aperture 46. Longitudinal axis 47 is perpendicular to upper leg 40 in the illustrated embodiment. Other embodiments contemplate that longitudinal axis 47 is angled or obliquely oriented with respect to upper leg 40 and longitudinal axis 25, e.g. so that aperture 46 extends distally toward intermediate portion 44 or toward mouth 54. Longitudinal axis 47 is offset from and parallel to longitudinal axis 25 toward mouth 54, and is aligned with lip 77 of foot portion 75 extending proximally from lower leg 75. In this arrangement, engaging member 48 is positioned to secure and direct rod 29 toward lower leg 42 and intermediate portion 44 and against saddle 26.

As shown in FIGS. 2 and 4, bone engaging portion 22 is attached in a particular embodiment to receiving portion 24 at a relatively lateral portion of lower leg 42 offset away from intermediate portion 44 more toward mouth 54 and foot portion 75. Bone engaging portion 22 may also be attached to receiving portion 24 at other parts of receiving portion 24. As one example, bone engaging portion 22 could be attached at a central portion of leg 42, i.e. an area approximately equidistant from intermediate portion 44 and foot portion 75. Bone engaging portion 22 could also be attached to leg 42 at an area relatively close to or aligned with intermediate portion 44.

Intermediate portion 44 joins aligned ends of legs 40 and 42. Taken together, legs 40 and 42 and intermediate portion 44 form substantially a C-shape, with channel 52 substantially to one side of intermediate portion 44. Receiving portion 24 further defines mouth 54 between legs 40 and 42. In a particular embodiment, mouth 54 forms an angled passage into channel 52 between foot portion 75 and distal surface 41 of upper leg 40 that is obliquely oriented to longitudinal axis 25. Mouth 54 and distal surface 41 extend obliquely to longitudinal axis 25 and slope in a proximal direction from longitudinal axis 25 to where mouth 54 exits receiving portion 24, as shown in FIG. 4.

In the illustrated embodiment, receiving portion 24 further includes a cavity 56 in lower leg 42 and intermediate portion 44. Cavity 56 is relatively shallow and hollow in a distal area 58 substantially corresponding to lower leg 42. Cavity 56 may be substantially cylindrical and extend from lower leg 42 and along a distal portion of intermediate portion 44 where it is in communication with blind hole 60, which opens into cavity 56.

In at least the distal area 58 of cavity 56, receiving portion 24 is provided with one or more ridges 62. Ridges 62 are provided to engage the exterior surface along the underside of saddle 26 when rod 29 is secured is channel 52 of bone anchor 20 with engaging member 48, such as shown in FIG. 4. In the illustrated embodiment, ridges 62 are positioned substantially perpendicular to the direction of rod 29 extending through channel 52. Alternatively, splines or other engaging structure can be provided on one or both of saddle 26 and the receiving portion 24 in the distal area 58 of cavity 56. In still other embodiments, a smooth surface interface is provided between the exterior surface of saddle 26 and receiving portion 24.

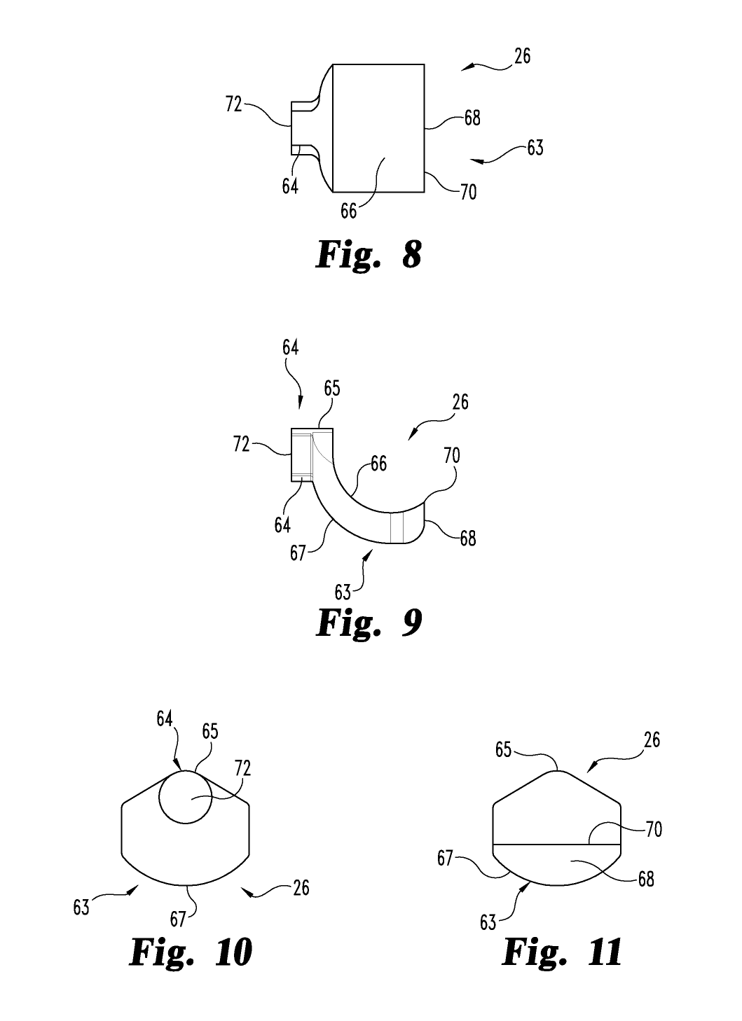

As further shown in FIGS. 8-11, saddle 26 includes a rounded seat portion 63 and a proximal post portion 64 along a proximal side 65 of saddle 26. Seat portion 63 has an internal curved surface 66 and an opposite external curved surface 67 along its under or distal side. Surfaces 66, 67 each extend to a second or lateral end 70, which includes a substantially flat abutting surface 68 extending between surfaces 66, 67. Internal surface 66, in a particular embodiment, has a substantially cylindrical shape which can have a radius the same as or similar to that of rod 29 positioned in channel 52 against internal surface 66. In one embodiment, the total arc of internal surface 66, from proximal surface 65 to abutting surface 68, is less than 180 degrees. In one particular embodiment, the total arc from proximal surface 65 to abutting surface 68 is 90 degrees. Other embodiments of saddle 26 may have an internal surface including an arc equal to or somewhat greater than 90 degrees.

Post portion 64 extends laterally from proximal side 65 of saddle 26 in a direction away from second end 70. In the illustrated embodiment, post portion 64 includes a lateral surface 72 that lies in a plane that is parallel to the abutting surface 68 defined at second end 70. Post portion 64 is substantially circular in cross-section and when viewed in the direction shown in FIG. 10.

Saddle 26 is positioned into cavity 56 of receiving portion 24 so that post portion 64 is within blind hole 60 of cavity 56, seat portion 63 is at least partially received within distal area 58 of cavity 56, and the abutting surface 68 at second end 70 abuttingly engages lip 77 of foot portion 75 extending proximally from lower leg 42. In order to accommodate this insertion, saddle 26 can be bent to a reduced size configuration by flexing second end 70 toward proximal end 65 about internal surface 66 until second end 70 is located so that it can be aligned with the inner side of lip 77 of foot portion 75. Saddle 26 is resilient to snap back toward its pre-insertion configuration when post portion 64 is received in hole 60 so that abutting surface 68 at second end 70 contacts lip 77 of foot portion 75. Saddle 26 is then captured in receiving portion 24.

In the captured position, saddle 26 can pivot or toggle with respect to receiving portion 24 with post portion 64 providing an axis about which saddle 26 rotates. Seat portion 63 generally rotates in a plane that is dictated by the placement of bone anchor 20 relative to the patient's anatomy. For example, if bone anchor 20 is attached to a pedicle of a vertebra with mouth 54 opening medially or laterally, saddle 26 rotates generally or substantially in or parallel to the sagittal plane. As another example, if bone anchor 20 is used with a lateral connector, such that mouth 54 of bone anchor 20 opens cephaladly or caudally, saddle 26 rotates in or substantially parallel to the coronal plane. In either case, saddle 26 rotates in a plane that defined by longitudinal axis 25 and rod 29 extending through channel 52 so that inner surface 66 is positioned or angled in receiving portion 24 in a manner that conforms with the angle of rod 29 through receiving portion 24, allowing inner surface 66 to maintain complete or substantially complete contact with rod 29 in receiving portion 24.

In the illustrated embodiment, engaging member 48 is a set screw that includes external threads adapted for engagement with threaded aperture 46 in upper leg 40. In one embodiment, engaging member 48 includes an imprint in or on a top or proximal surface adapted for engagement with a tool for tightening and/or loosening engaging member 48. The imprint may be internal, e.g. a hexagonal or hexalobed opening, or may be external, e.g. a hexagonal head. Engaging member 48 may further include a curved, pointed, conical or other surface 49 at its bottom or distal end. Such a surface engages rod 29 within receiving portion 24 as further described below. Engaging member 48 may be placed at least partially within threaded aperture 46 such that none or a very small amount of engaging member 48 extends into channel 52 to permit insertion of rod 29 through mouth 54. Alternatively, engaging member 48 may be left out of aperture 46 until rod 29 is inserted into receiving portion 24, and may then be inserted into aperture 46 and against rod 29, as further described below.

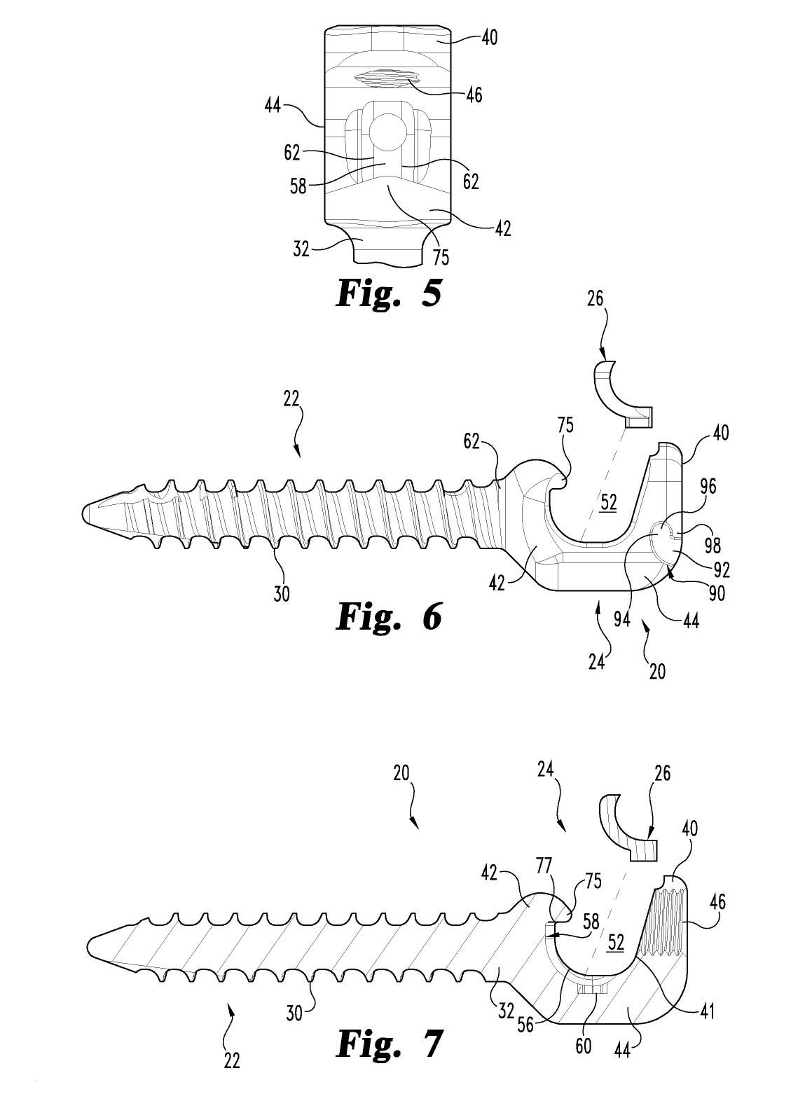

In addition to the features of the embodiments described above, receiving portion 24 may further include one or more indentations 90 for receiving a gripping or positioning tool. Indentations 90 are shown in one embodiment on either side of receiving portion 24, in an area in or adjacent to upper leg 40 and intermediate portion 44. It will be seen that indentations 90 could be in any part of receiving portion 24. As shown in FIG. 6, indentations 90 in the illustrated embodiment have an entry portion 92 and a holding portion 94. Holding portion 94 has a rounded or part circular portion 96 having a corner 98. A holding or gripping tool (not shown) having one or more rounded or circular protrusions at or adjacent to the end(s) of such a tool may be used. Such protrusion(s) may be inserted at entry portion(s) 92 and curved, angled or hooked around corner 98 into circular portion 96. In this manner, such a tool can be used to hold, position, manipulate or otherwise work on or with bone anchor 20.

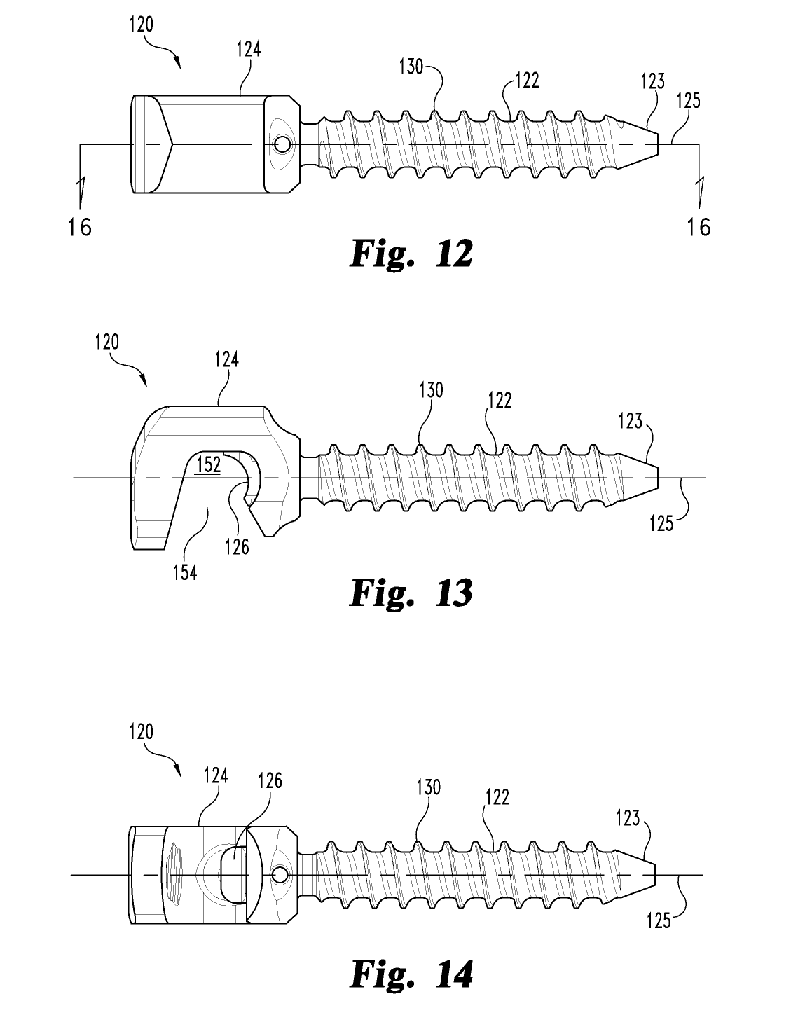

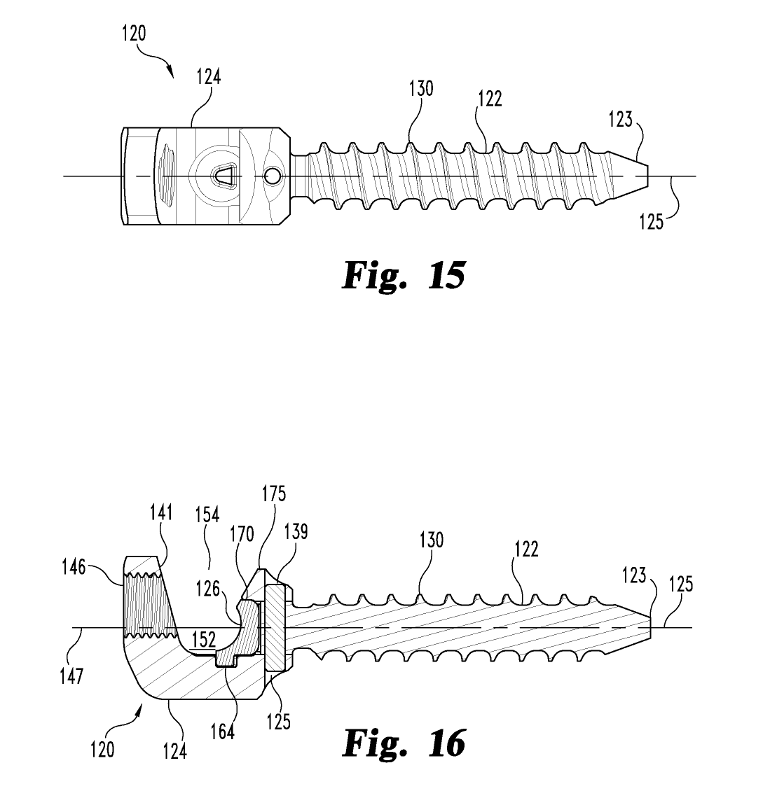

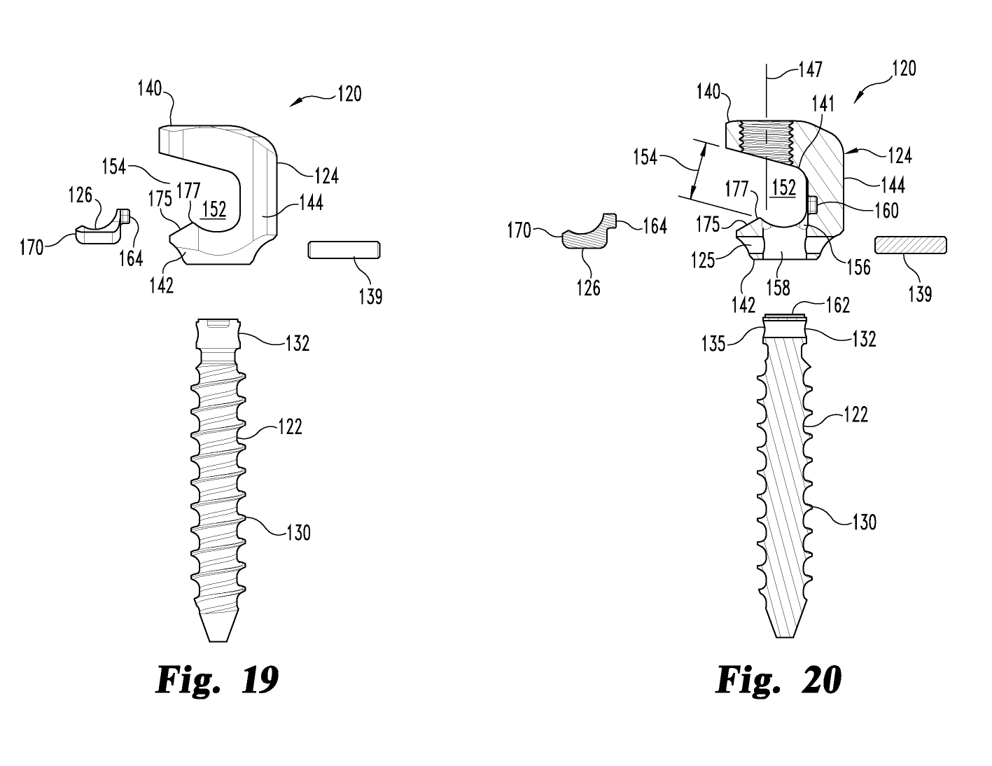

Another embodiment bone anchor 120 is shown in FIGS. 12-23. Bone anchor 120 includes an elongated bone engaging portion 122 extending from a distal end 123 along a longitudinal axis 125, a receiving portion 124 mounted at a proximal end of bone engaging portion 122, and a saddle 126 in receiving portion 124. Bone engaging portion 122 is shown as an elongated shaft having one or more threads 130 along at least a portion thereof. Thread 130 may be a cancellous thread with the shaft sized and configured for implantation into a vertebra or other bone. Bone engaging portion 122 and/or thread 130 may be self-tapping, self-drilling, continuous, intermittent, of multiple thread forms, or other appropriate configurations. A head 132 at a proximal end of bone engaging portion 122 extends between bone engaging portion 122 and receiving portion 124. Head 132 may be entirely proximal of thread 130, or may also include a portion of thread 130. In the illustrated embodiment, bone engaging portion 122 is integrally joined to head 132, as by unitary formation or construction or by solid attachment. Receiving portion 124 is formed as a separate component from bone engaging portion 122 and is mounted to bone engaging portion 122 during manufacture or in the operating room.

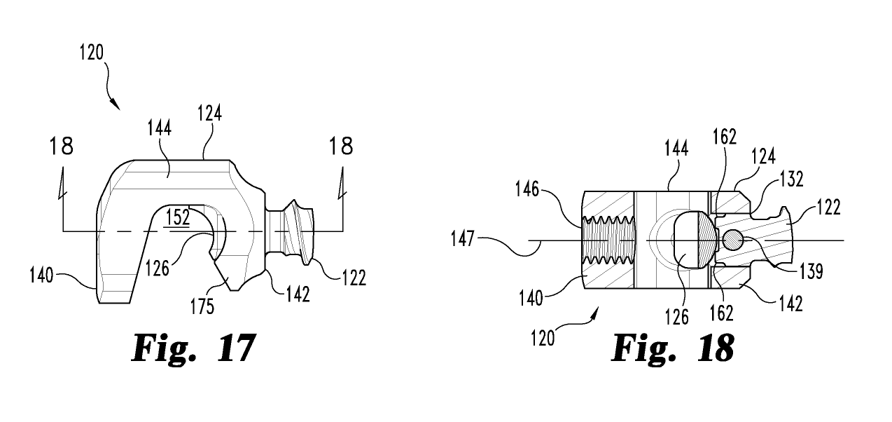

Referring now additionally to FIGS. 17-20, the depicted embodiment of receiving portion 124 is substantially C-shaped, having an upper leg 140, a lower leg 142 including foot portion 175 extending from one end thereof, and an intermediate portion 144 joining upper and lower legs 140, 142 opposite of foot portion 175. Other embodiments contemplate, for example, a U-shaped opening with a pair of intermediate portions extending proximally from lower leg 142. Receiving portion 124 defines a mouth 154 (FIG. 20) between upper leg 140 and foot portion 175 that is opposite intermediate portion 144. Mouth 154 opens into channel 152 extending through receiving portion 124, with channel 152 extending in an orthogonal relationship to longitudinal axis 125. Upper leg 140 has a threaded aperture 146 into which an engaging member, such as engaging member 48 can be threadingly engaged. Aperture 146 extends along a central longitudinal axis 147 defining the direction of advancement of engaging member 48 into aperture 146. Longitudinal axis 147 is perpendicular to upper leg 140 in the illustrated embodiment. Other embodiments contemplate that longitudinal axis 147 is angled or obliquely oriented with respect to upper leg 140 and longitudinal axis 125, e.g. so that aperture 146 extends distally toward intermediate portion 144 or toward mouth 154. Longitudinal axis 147 is offset from and parallel to longitudinal axis 125 toward mouth 154, and is aligned with lip 177 of foot portion 175 extending proximally from lower leg 175. In this arrangement, engaging member 48 is positioned to secure and direct rod 29 toward lower leg 142 and intermediate portion 144 and against saddle 126.

Intermediate portion 144 joins aligned ends of legs 140 and 142. Taken together, legs 140 and 142 and intermediate portion 144 form substantially a C-shape, with channel 152 substantially to one side of intermediate portion 144. Receiving portion 124 further defines mouth 154 between legs 140 and 142. Mouth 154 forms an angled passage into channel 152 between foot portion 175 and distal surface 141 of upper leg 140 that is obliquely oriented to longitudinal axis 125. Mouth 154 and distal surface 141 extend obliquely to longitudinal axis 125 and slope in a proximal direction from longitudinal axis 125 to where mouth 154 exits receiving portion 124, such as shown in FIG. 20.

Receiving portion 124 is shown in isolation in FIG. 21, and includes a cavity 156 in lower leg 142 and intermediate portion 144. Cavity 156 is relatively shallow and extends to distal receptacle 158 that opens through a distal side of lower leg 142. Cavity 156 extends from lower leg 142 and along a distal portion of intermediate portion 144 where it is in communication with blind hole 160, which opens into cavity 156. Receptacle 158 also opens into channel 152 so that ridges 162 of head 132 contact the distal surface of saddle 126 in cavity 156.

In at least the distal area of cavity 156, receiving portion 124 is provided with an axial through-hole or receptacle 158 in which head 132 of bone engaging portion 122 is positioned. Head 132 includes a hole 135 extending orthogonally to longitudinal axis 125, and receiving portion 124 includes lateral hole 125 extending laterally therethrough and through receptacle 158. A pin 139 extending through holes 125, 135 couples head 132 to receiving portion 124. Pin 139 can be press-fit in holes 125, 135 to fix bone engaging portion 122 and receiving portion 124 in position relative to one another. Other embodiments contemplate that receiving portion 124 is attached to bone engaging portion 122 by snap, ring or other suitable connector. Head 132 is positioned so that ridges 162 extending from the proximal side of head 132 are located in or adjacent to cavity 156 of receiving portion 124.

Ridges 162 extending from the proximal side of head 132 are provided to engage the exterior surface along the underside 167 of saddle 126 when rod 29 is secured in channel 152 of bone anchor 120 with engaging member 48. In the illustrated embodiment, ridges 162 are positioned substantially perpendicular to the direction of rod 29 extending through channel 152. Alternatively or additionally, ridges, splines or other engaging structure can be provided on one or both of saddle 126 and within receiving portion 124 in cavity 156. In still other embodiments, a smooth surface interface is provided between the exterior surface of saddle 126 and receiving portion 124. Since the ridges engaging saddle 136 are provided on head 132 of bone engaging portion 122, formation of saddle engaging ridges in receiving portion 124 are not required to fix saddle 126 in receiving portion 124.

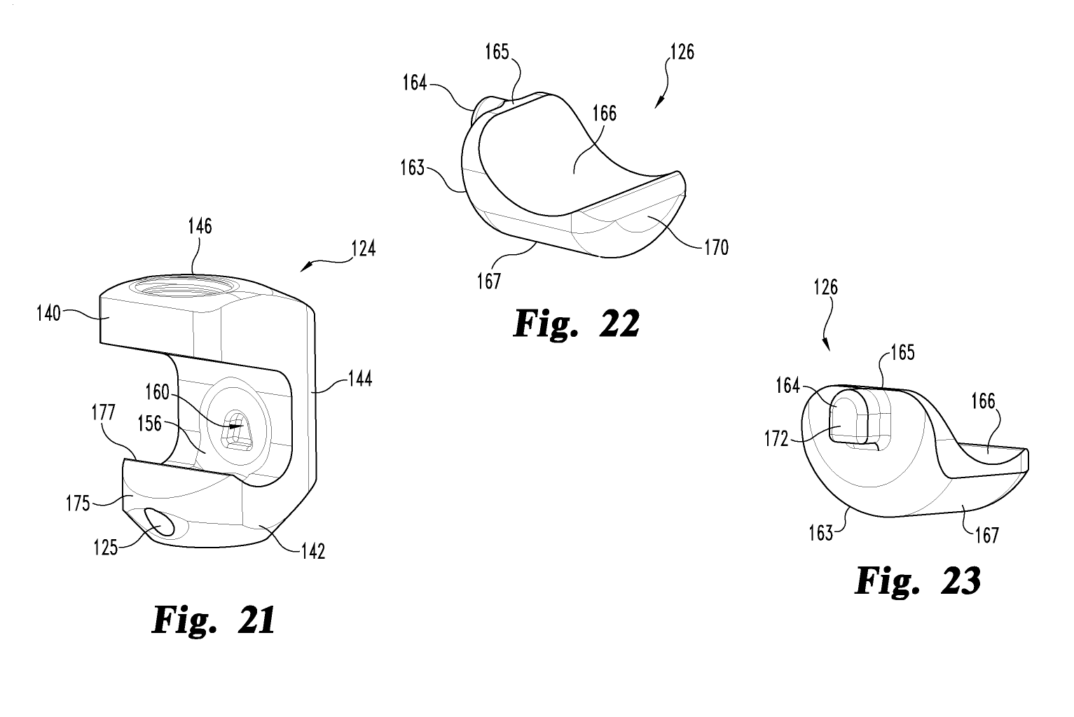

As further shown in FIGS. 22-23, saddle 126 includes a rounded seat portion 163 and a proximal post portion 164 along a proximal side 165 of saddle 126. Seat portion 163 has an internal curved surface 166 and an opposite external curved surface 167 along its under or distal side. Surfaces 166, 167 each extend to a second or lateral end 170. Internal surface 166, in a particular embodiment, has a substantially cylindrical shape which can have a radius the same as or similar to that of rod 29 positioned in channel 152 against internal surface 166. In one embodiment, the total arc of internal surface 166, from proximal surface 165 to end 170, is less than 180 degrees. Post portion 164 extends laterally from proximal side 165 of saddle 126 in a direction away from second end 170. In the illustrated embodiment, post portion 164 includes a lateral surface 172 that lies in a plane that is parallel to the second end 170. Post portion 164 is substantially hemi-ovular in cross-section. Saddle 126 is positioned into cavity 156 of receiving portion 124 so that post portion 164 is within the triangular-shaped blind hole 160 of cavity 156, and seat portion 163 is at least partially received within cavity 156 with second end 170 abuttingly engaging lip 177 of foot portion 175 extending proximally from lower leg 142. In order to accommodate this insertion, saddle 126 can be bent to a reduced size configuration by flexing second end 170 toward proximal end 165 about internal surface 166 until second end 170 is located so that it can be aligned with the inner side of lip 177 of foot portion 175. Saddle 126 is resilient to snap back toward its pre-insertion configuration when post portion 164 is received in hole 160 so that abutting surface 168 at second end 170 contacts lip 177 of foot portion 175. Saddle 126 is then captured in receiving portion 124. The hemi-oval shape of post portion 164 can pivot in the triangular shaped hole 160, and when one of the linear sides of post portion 164 contacts an angled side of the hole 160, pivoting movement of the saddle 126 is stopped.

In the captured position, saddle 126 can pivot or toggle with respect to receiving portion 124 with post portion 164 providing an axis about which saddle 126 rotates. Seat portion 163 generally rotates in a plane that is dictated by the placement of bone anchor 120 relative to the patient's anatomy. For example, if bone anchor 120 is attached to a pedicle of a vertebra with mouth 154 opening medially or laterally, saddle 126 rotates generally or substantially in or parallel to the sagittal plane. As another example, if bone anchor 120 is used with a lateral connector, such that mouth 154 of bone anchor 120 opens cephaladly or caudally, saddle 126 rotates in or substantially parallel to the coronal plane. In either case, saddle 126 rotates in a plane that defined by longitudinal axis 125 and rod 29 extending through channel 152 so that inner surface 166 is positioned or angled in receiving portion 124 in a manner that conforms with the angle of rod 29 through receiving portion 124, allowing inner surface 166 to maintain complete or substantially complete contact with rod 29 in receiving portion 124.

In use, a surgeon first prepares the surgical site as is generally known in the art, for example by making an open, minimally-invasive or other incision in the skin and subdermal tissues to obtain access to the desired surgical site, such as one or more vertebrae of the spinal column. Once access to a vertebra has been obtained, the surgeon prepares a hole in the vertebra or other suitable preparation to receive bone engaging portion 22, 122. Bone anchor 20, 120 is then introduced to the surgical site and threaded into or otherwise engaged to the vertebra. A holding tool (not shown) with protrusions connected to bone anchor 20 via indentations 90 can be used to hold and either begin to insert or completely insert bone anchor 20 into the hole in the vertebra. If bone engaging portion 22, 122 is self-tapping, then it will not be necessary to tap or otherwise thread the hole in the vertebra. Where a hook or other implant is employed for bone engaging portion 22, 122, the bone anchor 20, 120 is connected to the vertebra, as by hooking it around or otherwise in contact with a pedicle, process, lamina or other vertebral part.

When bone anchor 20, 120 is connected to a bone, a rod 29 can be maneuvered to the surgical site, contoured as may be desired, and then inserted into channel 52, 152 of receiving portion 24, 124 via mouth 54, 154. Rod 29 is placed in channel 52, 152 of receiving portion 24, 124 until it is adjacent or in contact with seat portion 63, 163 of saddle 26, 126. Rod 29 may be pressed against inner surface 66, 166 of saddle 26,126 which will result in a camming action to push seat portion 63, 163 of saddle 26, 126 relatively downward and/or around post 64, 164 so that inner surface 66, 166 aligns with the angle of rod 29 through channel 52, 152. Saddle 26, 126 is rotatable substantially in a plane with respect to receiving portion 24, 126 that includes longitudinal axis 25, 125 and rod 29, as discussed above, from a first orientation that is perpendicular to longitudinal axis 25, 125 to a non-perpendicular orientation relative to longitudinal axis 25, 125 to accommodate the angle of rod 29 through receiving portion 24, 124. Furthermore, the rotation of saddle 26, 126 is centered substantially around the axis of post portion 64, 164 of saddle 26, 126 in blind hole 60, 160. Once rod 29 and saddle 26, 126 are in the desired position with respect to receiving portion 24, 124, engaging member 48 is threaded down through upper leg 40, 140 of receiving portion 24, 124 and into contact with rod 29. Engaging member 48 forces rod 29 against seat portion 63, 163 of saddle 26, 126 forcing external surface 67, 167 of saddle 26, 126 against either lower leg 42 of receiving portion 24 and against one or more of the ridges 62 of receiving portion 24 or against the ridges extending from the proximal end of the bone anchor. In an embodiment in which engaging member 48 has a curved or conical bottom surface 49, such bottom surface 49 will tend to push the rod in a direction substantially toward intermediate portion 44, 144 and/or lower leg 42, 142 of receiving portion 24, 124. In an embodiment in which engaging member 48 has a pointed bottom surface 49, such point(s) may bite into, penetrate or deform the rod. The rod is then locked with respect to saddle 26, 126 and receiving portion 24, 124 and saddle 26, 126 is locked with respect to receiving portion 24, 124.

Additional bone anchors 20, 120 can be placed in adjacent or relatively distant bone tissue to connect to the same or additional rods. Further, other components, such as alternative screw or hook apparatus, clamps, connectors, or similar devices can be placed in connection with such rod(s) and such bone tissue(s). As may be desired by the surgeon or necessitated by the given trauma or other physical situation, bone growth materials, such as bone morphogenic protein (BMP), demineralized bone matrix (DBM), bone graft, or other substances may also be used in connection with parts of the structures described above so as to repair or correct the patient's physical situation.

For bone anchor 20, materials for engaging member 48, saddle 26, ridges 62 and/or the rod can be chosen so that some deformation or penetration of one part with respect to another may occur. For example, if engaging member 48 is a set screw that is provided with one or more points or edges on a bottom surface, such as curved or conical surface 49, such points or edges or the entire engaging member 48 can be made of a harder material than the rod so that engaging member bites into the rod as the engaging member 48 is tightened to lock the rod within receiving portion 24. As another example, if saddle 26 is of a softer material than ridges 62 of receiving portion 24, then ridges 62 can into external surface 67 of saddle 26 to lock the construct when the rod is secured to bone anchor 20.

For bone anchor 120, materials for receiving portion 124, saddle 126 and bone engaging member 122 can differ from one another to minimize the size of receiving portion 124 while enhancing the engagement of saddle 126 with ridges 162 of head 132 of bone engaging portion 122. For example, saddle 126 and bone engaging portion 122 and/or the rod can be chosen so that some deformation or penetration of one part with respect to another may occur. For example, if saddle 126 is of a softer material than ridges 162 of bone engaging portion 122, then ridges 162 can bite into external surface 167 of saddle 126 to lock the construct when the rod is secured to bone anchor 120. In addition, receiving portion 124 can be made from a high strength material that allows the size and footprint of receiving portion 124 to be minimized, reducing the invasiveness into the surrounding tissue when implanted. Bone engaging portion 122 can be made from a lower strength and lower cost material while maintaining the appropriate structural properties.

Bone anchor 20, 120 can be sized for placement at any level of the spine. Of course, it is understood that the relative size of the components of the assembly will be modified for the particular vertebra(e) to be instrumented. For example, components may be relatively larger for lumbar or sacral placement than those for cervical placement. Likewise, the relative dimensions of post portion 64, 164 of saddle 26, 126 and blind hole 60, 160 and/or the relative dimensions of the rod and receiving portion 24, 124, can be chosen to permit greater or lesser degrees of angulation of the rod relative to receiving portion 24, 124. Since saddle 26, 126 can be snapped or press fit into receiving portion 24, 124, hole 60, 160 can be a blind hole, which better maintains the structural integrity of receiving portion 24, 124 and intermediate portion 44, 144 than if a hole extending completely through intermediate portion 44, 144 were provided to accommodate insertion and engagement of saddle 26, 126 to receiving portion 24, 124. Other embodiments contemplate that intermediate portion 44, 144 includes a through-hole to receive post portion 64, 164.

The components described above may be formed of stainless steel or other suitable biocompatible materials, such as titanium, cobalt chrome, certain plastics or ceramics, and materials that permit bone ingrowth. Further, while the embodiment discussed above illustrates a bone screw, other bone anchors can be adapted to implement the features disclosed herein. For instance, as noted above, bone engaging portion 22, 122 could be the blade of a vertebral hook rather than a threaded element. It is also understood that while the illustrated embodiment of the invention engages a circular rod to the bone anchor, various longitudinal members are contemplated. For example, a non-circular rod, an elongated bar, tether, wire, cable, rigid, semi-rigid, flexible, elastic, or other suitable elongate member can be disposed within the channel of the receiving portion to be secured therein against saddle 26, 126. Such a rod or longitudinal member can be a part of a lateral connector or other member extending transversely to the central axis of the spinal column. The elongate member can include surfaces features such as knurling or threading, or can be smooth. Furthermore, other types of engaging members other than a set screw can secure the elongate member to the bone anchor, such as caps, nuts, slide-lock members, twist-lock members, and combinations thereof.

While the invention has been illustrated and described in detail in the drawings and foregoing description, the same is to be considered as illustrative and not restrictive in character. All changes and modifications that come within the spirit of the invention are desired to be protected.

What is claimed is:

1. A bone anchor apparatus for attachment of an elongated spinal rod to a vertebra, comprising:
a bone engaging portion having a threaded shaft extending along a longitudinal axis from a distal end to a proximal end of said threaded shaft;
a receiving portion including:
a lower leg connected to said proximal end of said bone anchor portion, said lower leg including a foot portion extending proximally from one end thereof;
an upper leg opposite said lower leg and extending transversely to said longitudinal axis;
an intermediate portion opposite said foot portion, said intermediate portion extending between said upper leg and said lower leg, said intermediate portion including a blind hole extending therein;
a channel extending through said receiving portion substantially perpendicular to said longitudinal axis into which the spinal rod can be placed, said receiving portion defining a mouth opposite said intermediate portion for introduction of the spinal rod into said channel, wherein said blind hole opens into said channel and said foot portion defines a lip that extends from said lower leg toward said mouth; and
a saddle connected to said receiving portion so that at least a portion of said saddle is within said channel of said receiving portion, said saddle including a proximal end with a post portion extending into said blind hole of said intermediate portion of said receiving portion, said saddle further including a seat portion extending between said proximal end and a lateral end, said lateral end being positioned in said channel adjacent to said lip of said foot portion with said post portion in said blind hole of said intermediate portion to secure said saddle in said channel, said seat portion defining a concavely curved inner surface along said channel.

2. The apparatus of claim 1, wherein said lip is offset from and extends parallel to said longitudinal axis and said lateral end is positioned in abutting engagement with said lip.

3. The apparatus of claim 1, wherein said upper leg includes an exterior indentation for receiving a tool portion, said indentation having an entry portion, a holding portion with a substantially circular part, and a corner between said holding portion and said entry portion, whereby a tool portion may be inserted through said entry portion in one direction and into said holding portion in another direction.

4. The apparatus of claim 1, wherein the spinal rod is positioned within said channel in contact with said inner surface of said saddle, and further comprising a set screw threaded through said upper leg and against the spinal rod to lock the spinal rod, said saddle, and said receiving portion together.

5. The apparatus of claim 1, wherein said post portion and said blind hole are circular in cross-section.

6. The apparatus of claim 1, wherein said post portion is hemi-ovular in shape and said blind hole is triangular in shape.

7. The apparatus of claim 1, wherein said saddle is rotatable in said receiving portion about said post portion in said blind hole to adjust said concavely curved inner surface from a perpendicular orientation relative to said longitudinal axis to non-perpendicular orientations relative to said longitudinal axis.

8. The apparatus of claim 1, wherein said saddle is bendable to a reduced size configuration to position said saddle in said channel with said post portion in said blind hole and said lateral end adjacent to said foot portion, said saddle being resilient to contact said lateral end with said lip of said foot portion after said saddle is positioned in said channel to maintain said post portion in said blind hole.

9. The apparatus of claim 1, wherein said bone anchor portion includes a head at a proximal end thereof and said receiving portion includes a through-hole through said lower leg that extends from said passage to a distal side of said lower leg, and said head is positioned in said through-hole and said receiving portion is fixed to said bone anchor portion.

10. The apparatus of claim 9, wherein said head includes ridges extending proximally therefrom that contacts a distal surface of said saddle opposite said concavely curved inner surface of said saddle.

11. A bone anchor apparatus for attachment of an elongated spinal rod to a vertebra, comprising:
a bone engaging portion extending along a longitudinal axis between a distal end and a proximal end;
a receiving portion having a lower leg extending from said proximal end of said bone engaging portion, said receiving portion including at least one intermediate leg extending from a first side of said lower leg along said longitudinal axis and a foot portion extending proximally along said longitudinal axis from a second side of said lower leg opposite said intermediate portion, said receiving portion including a channel defined at least in part by said lower leg, said intermediate portion and said foot portion, said channel extending substantially perpendicular to said intermediate portion and said longitudinal axis, said receiving portion further including a cavity in said lower leg extending from said foot portion and across said lower leg to a blind hole extending into said intermediate portion, said cavity and said blind hole opening into said channel; and
a saddle including a post portion and a seat portion, said post portion projecting laterally from said seat portion adjacent to a proximal end of said saddle, said seat portion extending from said post portion along a substantially cylindrically shaped concave inner surface to a second end of said saddle, wherein said seat portion is positioned at least in part in said cavity with said second end in abutting engagement with said foot portion and with said post portion in said blind hole.

12. The apparatus of claim 11, wherein said saddle is bendable to a reduced size configuration to position said saddle in said channel with said post portion in said blind hole and said lateral end adjacent to said foot portion, said saddle being resilient to contact said lateral end with a lip of said foot portion after said saddle is positioned in said channel to maintain said post portion in said blind hole.

13. The apparatus of claim 12, wherein said lip is offset from and extends parallel to said longitudinal axis.

14. The apparatus of claim 11, further comprising:
an upper leg extending from a proximal end of said intermediate portion across said longitudinal axis, said upper leg including a threaded aperture extending therethrough along said longitudinal axis; and
an engaging member connected to said upper leg of said receiving portion in said threaded aperture.

15. The apparatus of claim 14, wherein said engaging member and said seat portion of said saddle are adapted to contact the spinal rod when positioned in said channel to lock the spinal rod, said seat portion, and said receiving portion with respect to each other.

16. The apparatus of claim 11, wherein said receiving portion includes at least one ridge extending along said cavity, said ridge adapted to contact said saddle along a convexly curved surface of said saddle that is opposite of said inner surface of said saddle.

17. The apparatus of claim 11, wherein said bone engaging portion includes an elongated threaded shaft extending along said longitudinal axis and a head at a proximal end of said shaft, and said receiving portion includes a receptacle receiving said head, said head being fixed in said receptacle.

18. The apparatus of claim 11, wherein said saddle includes a convexly curved outer surface opposite of said inner surface extending between said proximal end and said second end, said post portion extending laterally from said seat portion at said proximal end of said saddle in a direction opposite of said second end, wherein said post portion includes a lateral surface that is parallel to an abutting surface at said second end of said saddle that contacts said foot portion.

19. The apparatus of claim 11, further comprising:
an upper leg extending from a proximal end of said intermediate portion across said longitudinal axis; and
a mouth opening into said channel, said mouth being defined between said foot portion and an end of said upper leg that is opposite said intermediate portion, wherein said mouth extends outwardly and proximally from said channel in an oblique orientation to said longitudinal axis.

20. A bone anchor apparatus for attachment of an elongated spinal rod to a vertebra, comprising:
a bone engaging portion extending along a longitudinal axis between a distal end and a proximal end;
a receiving portion having a lower leg extending from said proximal end of said bone engaging portion, said receiving portion including at least one intermediate leg extending from a first side of said lower leg along said longitudinal axis and a foot portion extending proximally along said longitudinal axis from a second side of said lower leg opposite said intermediate portion, said receiving portion including a channel defined at least in part by said lower leg, said intermediate portion and said foot portion, said channel extending substantially perpendicular to said intermediate portion and said longitudinal axis, wherein said intermediate portion includes a blind hole opening into said channel; and
a saddle including a post portion and a seat portion, said post portion projecting laterally from said seat portion adjacent to a proximal end of said saddle, said seat portion extending from said post portion along a substantially cylindrically shaped concave inner surface to an abutting surface at a second end of said saddle, wherein said saddle is bendable to a reduced size configuration to position said saddle in said channel with said post portion in said blind hole and said abutting surface adjacent to said foot portion, said saddle being resilient from said reduced size configuration to contact said abutting surface with a proximally extending lip of said foot portion after said saddle is positioned in said channel to maintain said post portion in said blind hole.
